(19) United States
(12) Patent Application Publication
(10) Pub. No.: US 2008/0238174 A1
(43) Pub. Date: Oct. 2, 2008

(54) Traveler's head support and optional jaw support device for onboard resting

(76) Inventor: Matthew Damian Cinquanta, Sherman Oaks, CA (US)

Correspondence Address: MARC E. HANKIN, ESQ., 11414 THURSTON CIRCLE, LOS ANGELES, CA, 90049, US

(21) Appl. No.: 11/729,386

(22) Filed: Mar. 29, 2007

(51) Int. Cl. A61F 5/00

(52) U.S. Cl. 297/397; 24/442

(57) ABSTRACT

Disclosed is a head support device for comfortably supporting a person's head while the person is seated in a seat that may or may not include a headrest. The support device includes a headband, a jaw band, and a securing seat-back piece. The securing seat-back piece is removably fixable to the seat and/or headrest to support the person's head against the seat. The optional jaw band is worn around the person's chin and upwardly attached to the headband. The support system restrains the seated person's head from falling forward and/or moving laterally, and restrains the user's jaw from dropping and the mouth falling open.

### FIELD OF INVENTION

This invention relates generally to head supports. More specifically, this invention relates to devices for supporting the head and jaw comfortably and firmly while at rest, for the purpose of sleeping and/or resting while traveling in aircraft, trains, buses, cars, and/or other suitable transportation vehicles that are equipped with passenger seats.

### BACKGROUND

For many people, the inability to sleep or rest as a passenger on an aircraft, train, bus, car, and/or other suitable transportation vehicle is a tiring, uncomfortable, stressful and physically taxing experience. The inability to sleep while traveling can be directly related to excessive tiredness, physical discomfort and emotional distress. Also, it can lead to detrimental physiological conditions that are often suffered by travelers, such as severe neck and back strain, which often leads to subluxations of the spinal column that require chiropractic treatment in order to recover. The excessive leaning and jerking of the head and the cramping and straining of the neck and back muscles that occur while a traveler attempts to rest but cannot do so comfortably is easily avoidable when said invention is used. Use of the invention can also dramatically reduce the incidence of jet lag, from which travelers have been known to suffer dramatically and have even documented severe physiological, psychological and emotional distress from the continuous lack of proper sleep when traveling frequently between time zones.

The invention can also be implemented in a variety of occupations that require one to engage in rest breaks while on the job or when traveling. Occupations such as pilots, flight crews, and truck and bus drivers have reported experiencing high levels of sleep deprivation and/or the inability to rest when given the chance because of the lack of a comfortable means to do so. Additionally, the device's parts or sections are easily laundered, and more importantly, are easily stowed in a person's luggage or even pockets, taking up little more space than a pair of socks.

There have been a number of various concepts and designs pertaining to supporting a person's head while seated and/or traveling. However, each of the previous art relies on cumbersome, impractical, unsafe and inconvenient methods. Additionally, the effectiveness of each design depended largely on not only its own design but also upon the design or dimensions of the traveler's seat. Furthermore, they do not display a concern for the traveler's physical body, do not address the safety and security issues and the restrictions of today's airlines or the level of inconvenience to the traveler for traveling with such devices. Most claim to primarily keep a person's head in place and have proposed doing so by the use of contraptions that either render the person immobile and probably uncomfortable or achieve head immobility through rigid, uncomfortable, and unsafe design.

The previous conceptual headrest designs lack focus on comfort, proper support, safety and practicality. Most rely on an immobilizing and unsafe means of head support involving both head and seat straps, which wrap around both the seat and the user's head. Not only is this type of design impractical because of excess strap length that may need to be overlapped, but it is also unsafe in that it leaves the user's head in an immobilized, rigid position and does not allow free, voluntary movement. This is not only unsafe in the event of sudden, sharp or abrupt vehicle or aircraft movement, but it also prevents the person from easily engaging in eating, conversation, or other tasks that require at least a small degree of mobility. This, in turn, reduces the comfort and ease of using such a device. The strap also has to extend around the seat, which will likely prevent the food tray of the next passenger form being used, but may also restrict the viewing of TV or LCD screens of today's technologically-advanced airline seats. Additionally, many of the previous designs rely on small hooks, buckles or other means of metal or hard material fastening systems to secure the device into place. This not only adds to the reduced ease of packing and traveling with the device, but also brings into concern the safety issues surrounding today's modes of travel. Any object that could be considered a potential weapon against a passenger or crewmember may be restricted from onboard usage.

There are also a number of portable head support devices, such as small neck cushions, U-shaped neck and collar-type headrests and inflatable neck pillows. These devices provide limited support and do not prevent the user's head from falling forward and partially sideways. They do not support the head entirely and serve more as neck cushions, not head supports.

Each of the previous conceptual designs provide only limited support of the head and fail entirely to address the need for a passenger's jaw to be supported, at least optionally, during sleep. With the previous art, if sleep does occur, the head is able to sway or fall forward and the jaw is allowed to fall open, thus causing the user to experience the sudden awakening to embarrassing, head jerking and mouth-hanging-open moments. With these devices, the risk of assuming that adequate support is being provided when it indeed may not be may constitute a physical health hazard to the user.

### SUMMARY OF THE INVENTION

The present invention is unique in that: it is comprised from two, relatively small pieces of simple, comfortable, elastic headband-type material, and one small, quick and easy to use, disposable hook-and-loop type closure material, or the like. The adhesive surfaces upon each of the invention's parts serve as an easy, convenient way to put on and take off the device. Furthermore, the device's third piece is a disposable, easily transportable piece of hook-and-loop type closure-like material and is utilized to serve as the means to secure the user's head against the seat, instead of crude, impractical straps and tightening devices that wrap around the seat an/or head as in other designs. The invention strategically utilizes a small hook-and-loop type closure attachment piece to serve as its anchoring piece, which amplifies the invention's ease of use and convenience. Furthermore, each anchoring hook-and-loop type closure piece (of several to be supplied with the invention or which can be easily obtained inexpensively from the general marketplace) is easily disposable after each use. The invention does not require tightening or loosening adjustments, as it forms elastically to the dimensions of the user's head and jaw. The present invention addresses and fulfills the need for a small, convenient, practical device to provide comfortable head and jaw support for a seated passenger. It is safe, lightweight and easily stowed, used, and laundered and is airline safety regulations friendly. The invention is the only device that provides comfortable head and jaw support while seated, and conveniently eliminates the concerns of neck and back discomfort and embarrassing mouth open moments that stem from the inability to sleep comfortably while traveling. Continuing, the device provides the user with the ease and convenience of being able to eat, talk and even turn the head voluntarily with the device in place. And finally, the ease of disengaging the device from the seatback with only slight effort emphasizes the invention's convenience and comfort.

The problems with the prior art are overcome by the present invention in that the present invention provides a head support that does not require tightening and loosening straps. Additionally it does not require using the full dimensions of a seatback in order to be in place and used, does not immobilize the user's head in a rigid, restricting manner, does not jeopardize the user's safety with rigid means of support, does not fail to address the safety concerns of today's travel industry, does not restrict the normal use of food trays and LCD screens on many of today's passenger seats, and does not prevent the user from voluntarily moving or turning their head. Furthermore, the problems of the prior art are overcome by the present invention in that the present invention is safe, even during sudden vehicle or aircraft movement, is quickly and easily applied and removed, and fully addresses the need for a seated passenger's jaw to be supported, at least optionally. The present invention is also safe and lightweight, and is easily stowed, easily used, easily laundered and is compliant with today's strict travel safety regulations.

A self-adjusting head and jaw support sleeping device, the present invention includes a resilient, self-tightening head and jaw band assembly able to be fastened at self-adhering junctures, and is able to adapt to any size or dimension of a user's head. The present invention is universally affixed to any passenger seat material within the headrest or seat surface area by the adhesive-backed hook-and-loop type closure material piece, which serves as the anchoring piece

The anchoring piece is attached to any passenger seat surface area where the passenger's head comes into contact with the seat to form a head support for restraining the user's head against forward and/or lateral side-to-side movement relative to the position of the person's head against the headrest.

The headband is worn around the user's head and is secured into position by its elastic resiliency, which allows the headband to comfortably form to the size and shape of the user's head. The headband piece may be universally sizeable due to its elastic composition.

The jaw support piece is attached at each of the temporal sides of the headband piece and is worn around the chin, thus supporting the jaw and preventing it from falling open during sleep, yet allowing easy, voluntary movement of the mouth and jaw.

In one form of the present invention the resilient headband includes material of cotton-covered, elastic material, with its hook-and-loop type closure fastening surfaces remaining highly adhesive to its opposing contact surfaces on the attachment points of each adjoining piece throughout the invention's operating life. The contact surfaces remain highly adhesive and grip strongly in a bond to each other irrespective of the number of times the surfaces are brought into or removed from contact with each other.

In one form of the present invention the resilient jaw-band includes material of cotton-covered, elastic material, with its hook-and-loop type closure fastening surfaces remaining highly adhesive to its opposing contact surfaces on the attachment points of each adjoining piece throughout the invention's operating life. The contact surfaces remain highly adhesive and grip strongly in a bond to each other irrespective of the number of times the surfaces are brought into or removed from contact with each other.

In one form of the present invention the adhesive-backed seat attachment/anchoring hook-and-loop type closure piece includes material of semi-resilient plastic with an adhesive backing on its back surface and hook-and-loop type closure upon its front surface. Its hook-and-loop type closure fastening surface remains highly adhesive to its opposing contact surfaces on the attachment point of the headband piece throughout the invention's operating life. The contact surface remains highly adhesive and grips strongly in a bond to each other irrespective of the number of times the surfaces are brought into or removed from contact with each other. Its adhesive backing provides a strong, anchoring attachment to the passenger seat for one to two uses, providing easy disposal after use and the ease of utilizing one of several identical replacement pieces during future travel that are supplied with the invention, or that can be easily and inexpensively obtained. This feature of said invention eliminates the need to use crude, cumbersome, rigid, unsafe and inconvenient strap like assemblies to secure the user's head as in many of the previous conceptual designs.

The present invention is a head and jaw band assembly and seat attachment design that is universally adaptable to any passenger seat and person's head. The manner in which the present invention comfortably and firmly supports the head and jaw of a seated person while at rest, relative to the position of the person's head against the passenger seat, is also new and unique.

A feature of the resilient headband and jaw-band pieces described herein is a cotton or terry cloth surface that surrounds each piece's elastic composition, thus providing a comfortable surface area around its entirety at which the user can allow comfortable contact with the forehead, face and jaw. This feature, along with the durability of the material of the head and jaw band, allows for easy, comfortable use and easy laundering and stowing.

### BRIEF DESCRIPTION OF THE DRAWINGS

FIG. 1 is a perspective view of the present invention.

FIG. 2 is a perspective view of the present invention showing the points of attachment to a standard vehicle seat.

FIG. 3 is top elevation view of the present invention showing the points of attachment to a standard vehicle seat.

FIG. 4 is a top elevation view of the present invention as connected to a standard vehicle seat.

FIG. 5 is a side view of the present invention in use.

FIG. 6 is a perspective view of the present invention in use.

FIG. 7 is a top elevation view of an alternative embodiment of the present invention including an intervening headrest.

### DETAILED DESCRIPTION OF THE DRAWINGS

In FIG. 1 the present invention 50 is shown; an example of a head and jaw support system for a seated, sleeping and/or resting person, according to this invention, is shown. FIG. 1 indicates in a perspective view all of the parts of the invention, separated, ready to be fitted into place on the head of a seated person or traveler.

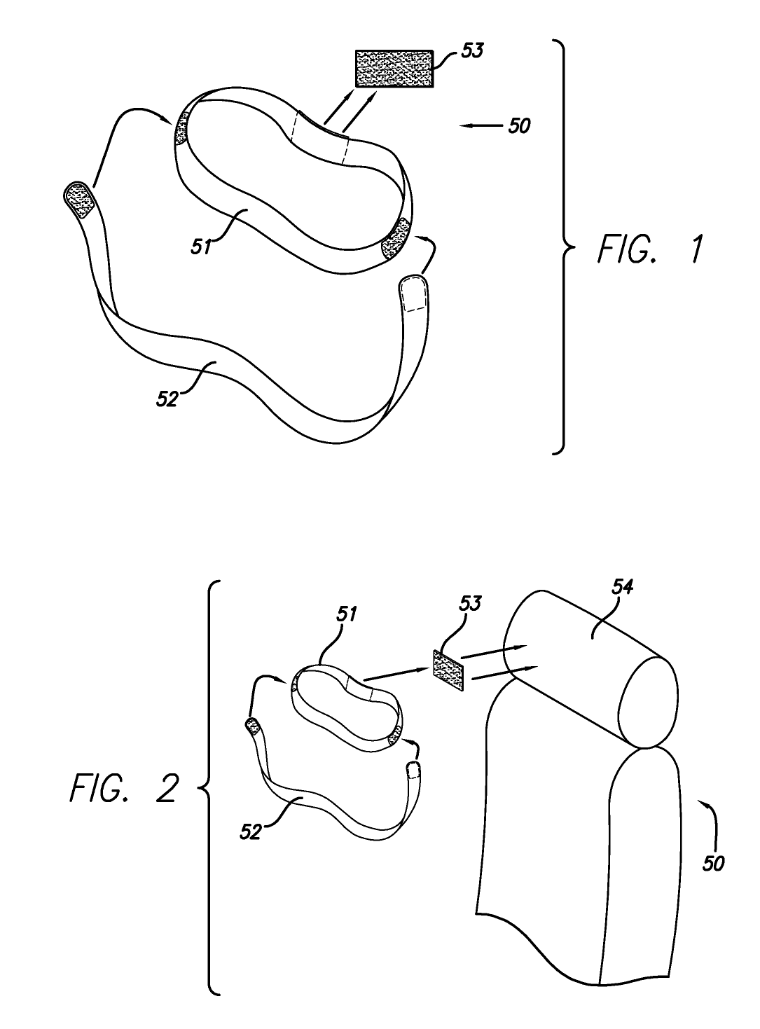

In FIG. 2, the operational fitting application of such a head and optional jaw support sleeping and/or resting device is shown. FIG. 2 indicates in a perspective view all of the parts of the invention, separated, ready to be fitted into place on a common passenger seat within the headrest surface area.

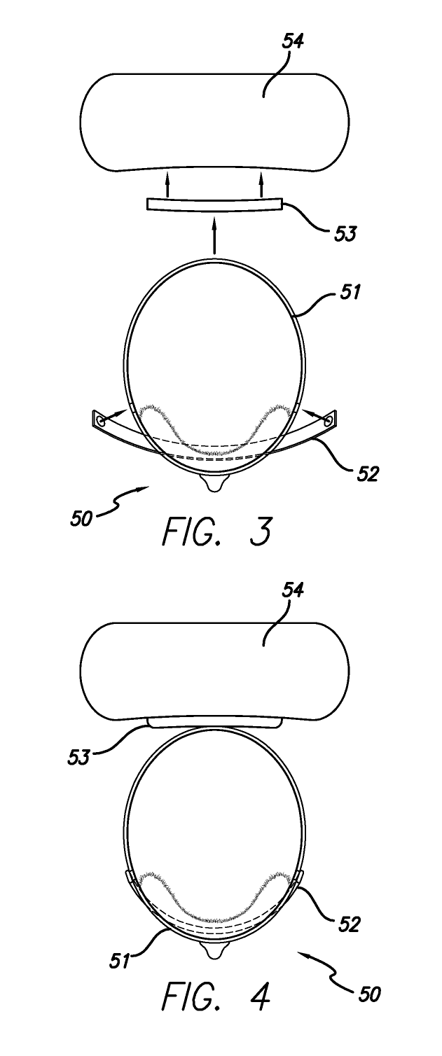

FIG. 3 shows in top elevation, about to be fitted into place on a common passenger seat with a headrest type surface area. FIG. 3 shows the top of a common passenger seat with a headrest surface area.

FIG. 4 shows in top elevation, the operational fitting application of such a head and jaw support sleeping/resting device, fully fitted into place on a common passenger seat with a headrest surface area.

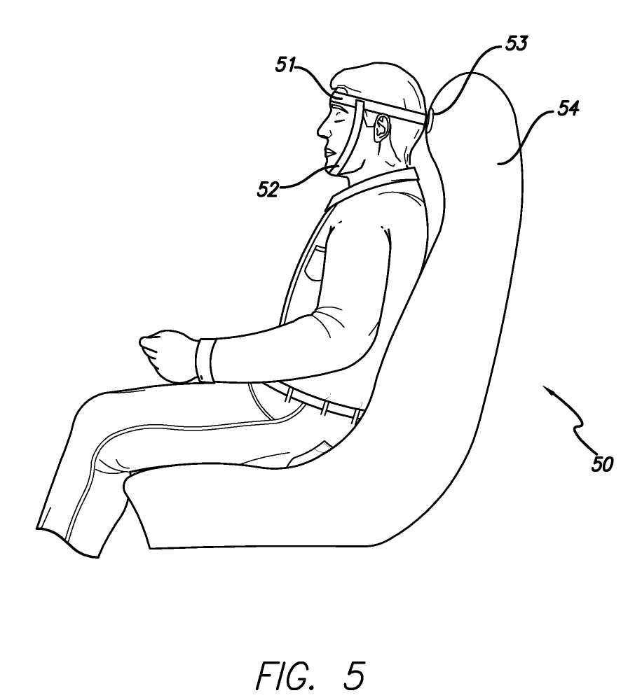

FIG. 5 shows the fully fitted application of such a head and jaw support sleeping and/or resting device assembly. In FIG. 5, the full fitting also includes a side view of a human figure and the full fitting of the head and jaw support sleeping and resting device assembly connected to the seat and holding the head and jaw of a human figure. FIG. 5 indicates all parts having been fitted into place on a common passenger seat with a headrest area.

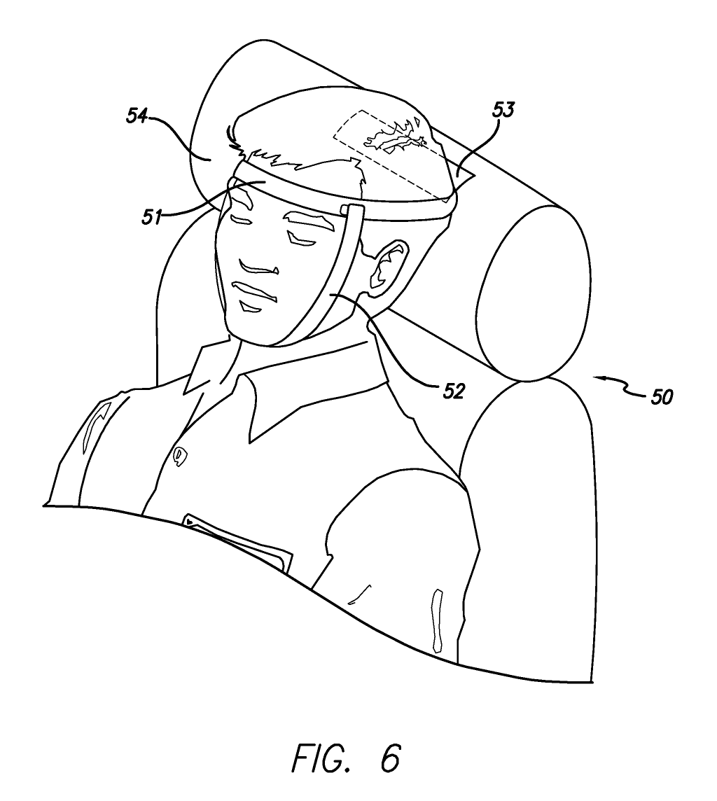

FIG. 6 shows the fully fitted application of such a head and jaw support sleeping and/or resting device assembly in perspective view. In FIG. 6, a perspective view of the common passenger seat with a headrest surface area and how the fully fitted application of such a head and jaw support sleeping and/or resting device assembly is to be effected is shown.

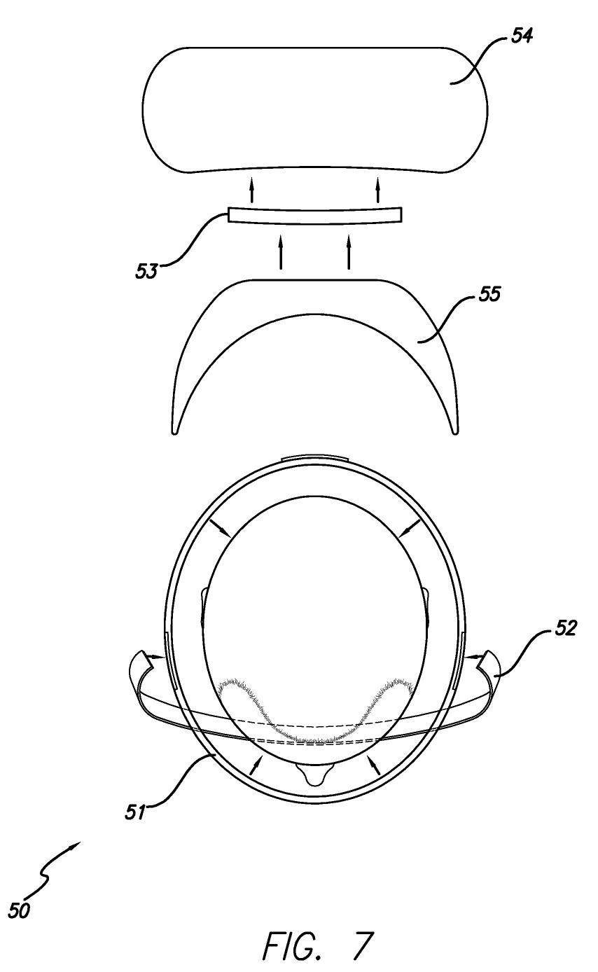

FIG. 7 shows a top elevation view, with all parts separated and to be fitted into place on a common passenger seat with a headrest type surface area, but with an alternative embodiment U-shaped piece of resilient material 55 removably affixed between the headband 51 and the adhesive-backed hook-and-loop type closure piece 53.

Referring to FIG. 1, it can be seen by the three dimensional drawing that the present invention 50 includes a circular headband, an elongated jaw-band and an adhesive-backed hook-and-loop type closure piece, thus comprising three separate sections, a headband, a jaw band, and a securing, seat-back hook-and-loop type closure piece. The elastic headband 51 and the elongated, elastic jaw-band 52 are preferably made of resilient, cotton or terrycloth covered elastic material, while the adhesive-backed seatback hook-and-loop type closure piece 53 is preferably made of semi-resilient plastic with a strong adhesive backing and a hook-and-loop type closure front surface.

The headband 51, the jaw-band 52 and the adhesive-backed hook-and-loop type closure piece 53 preferably have contact surfaces that are highly adhesive to their opposite sides. The headband 51 and the jaw-band 52 form the jaw support assembly of the present invention. The headband 51 and the adhesive-backed hook-and-loop type closure piece 53 form the head support assembly of the present invention. The adhesive-backed hook-and-loop type closure piece 53 and the passenger seat headrest surface area 54 form the anchoring assembly of the present invention.

At the appropriate junctures, the adhering surfaces of the headband 51 and the jaw-band 52 and adhesive-backed hook-and-loop type closure piece 53 are laid upon each other. The headband 50 and the jaw-band 51 form a strong bonding adhesion and provide a secure and comfortable jaw support assembly. The headband 51 and the adhesive-backed hook-and-loop type closure piece 53 form a strong adhesion and provide a secure head support assembly. The adhesive backing of the adhesive-backed hook-and-loop type closure piece 53 and the passenger seat headrest surface area 54 form a strong adhesion and provide a secure anchoring assembly.

The headband 51 is laterally placed around the head of the seated person, and is removably attached to the headrest surface area of the passenger seat via the adhesive-backed hook-and-loop type closure piece 53 as shown in FIG. 2. The optional jaw-band/jaw support 52 is laterally and upwardly attached to the temporal sides of the headband 51 as shown in FIG. 2. The adhesive-backed hook-and-loop type closure piece 53 is frontally attached to the passenger seat material within the headrest area of the passenger seat 54 as shown in FIG. 2. This arrangement allows the present invention 50 to form a secure head support 51 and jaw support 52 on whatever common passenger seat applied thereto by the present invention 50.

The present invention 50 includes jaw-band 52 which when so bonded to the headband 51 provides a loop to be comfortably fitted around the chin. The loop being so formed in this fashion so as to comfortably fit around the chin and thereby holding the jaw comfortably closed and relaxed for the purpose of sleeping, resting or relaxing. The headband 51 and the adhesive-backed hook-and-loop type closure piece 53 form a head support assembly.

In one form of the present invention 50, the head and jaw support assembly (the headband 51, the jaw-band/jaw support 52 and the adhesive-backed hook-and-loop type closure piece 53) has universal capability of being affixed to any passenger seat either existing or proposed. The headband 51 and jaw-band 52 form the jaw support assembly, while the headband 51 and adhesive-backed hook-and-loop type closure piece 53 form a head support assembly.

Referring to FIG. 2, it can be seen that the present invention 50 may be affixed to any common passenger seat of any seatback material existing or proposed, most commonly of fabric or leather composition in the headrest surface area of the passenger seat. The present invention thereby requires no adjustment or alteration in order to implement use.

FIG. 3 shows the present invention 50 in use. The adhesive-backed hook-and-loop type closure piece 53 can be affixed to any passenger seat with a headrest surface area by removing its protective backing and adhering its highly adhesive back surface directly upon the material within the headrest surface area of the passenger seat 54. Thus, a secure anchoring point for the headband 51 is formed. The adhesive-backed hook-and-loop type closure piece 53 is removably attached to the passenger seat, leaving no adhesive residue or damage upon the seat's material after use. The headband 51, when placed around the person's head as a typical head sweat-band, may be secured to the adhesive-backed hook-and-loop type closure piece 53 via the adhering hook-and-loop type closure surfaces upon each and thereby provide a secure, comfortable head support. The headband 51 is removably attached to the adhesive-backed hook-and-loop type closure piece 53. The jaw-band 52, when placed around the person's chin as a typical helmet jaw-strap would be, may be secured to the headband 51 via the adhering hook-and-loop type closure surfaces upon each and thereby provide a secure, comfortable jaw support. The optional jaw-band 52 is removably affixed to the headband 51. The support being formed in this fashion so as to comfortably place the user's chin and jaw in a secure position and thereby holds the chin comfortably closed when at rest, for the purpose of sleeping and/or resting with the mouth remaining comfortably closed.

FIG. 4 is a top elevation view of the present invention 50 in use. FIG. 4 shows the adhesive-backed hook-and-loop type closure piece 53 as attached to the surface area within the headrest area of the passenger seat 54, and the headband is shown as worn around the person's head and as attached to the adhesive-backed hook-and-loop type closure piece 53. The optional jaw-band support 52 is partially shown in FIG. 4.

Referring to FIG. 5, it can be seen by the side view drawing, that the present invention 50 may be affixed to any common passenger seat 54 with a headrest surface area. The head and jaw support assembly (the headband 51, the jaw-band 52 and the adhesive-backed hook-and-loop type closure piece 53) may be varied as to the placement of each of their adhering, bonding attachment points, so as to provide some degree of variation to fitting preferences, although the invention itself requires little variance of its attachment points to ensure comfortable, effective use. Particularly, the variation of height at which the person's head is in contact with the passenger seat is fully addressed by the ease of placement of the adhesive-backed hook-and-loop type closure piece 53 anywhere upon the passenger seat's surface area. Thus, the user's head can be brought into a secure attachment point against the headrest at which the person is most comfortable. The common passenger seat 54 may be any seat with a headrest area. The common passenger seat 54 need not be shaped as shown in FIG. 5.

Referring to FIG. 6, it can be seen by the perspective view drawing, that the present invention 50 may be affixed to any common passenger seat 54 with a headrest surface area by way of the adhesive-backed hook-and-loop type closure piece 53 anywhere upon the passenger seat's surface area. The optional jaw-band 52 is shown affixed to the headband 51.

By the use of a head and jaw support device of this type, the head and jaw of a seated passenger may be comfortably and securely held in a normal, postural position while at rest. The head and jaw being held comfortably and securely in this manner will prevent the passenger from having to rely on neck muscle and tendon structure to support the head while at rest, preventing it from involuntarily moving, falling and jerking, as well as preventing the mouth from falling open. This presents the opportunity for the traveler to conveniently use the invention to support his or her head comfortably and securely enough to promote and enjoy restful, comfortable travel. Thus, the experience of fatigue, exhaustion, neck and back cramps, injuries, discomfort and even embarrassment is eliminated, and the user can further enjoy the use of such a device through its ease of use, transport and laundering capabilities.

An alternative embodiment of the present invention 50 is to include a U-shaped piece of plastic, rubber, or other resilient material 55 into which the back of the user's head is placed, with the piece's rear, flat surface equipped with an adhering hook-and-loop type closure surface, and said surface being secured into place against the anchoring adhesive-backed hook-and-loop type closure piece 53. The interior surface of said U-shaped piece also being an adhering hook-and-loop type closure surface that removably bonds to the hook-and-loop type closure surface of the headband 51. Thus, the user's head is further prevented from turning or pivoting while the user is at rest.

Furthermore, the adhesive-backed hook-and-loop type closure piece 53, while attachable to the U-shaped piece of plastic, rubber, or other resilient material 55, may be an integral part of the seat design and/or the seat covering materials design itself or attached thereto. The adhesive-backed hook-and-loop type closure piece 53 may be an integral part of the seat 54 design. The adhesive-backed hook-and-loop type closure piece 53 may be removably affixed or an extension of the seat 54 or seat materials and/or fastened to the seat 54 or seat cover materials by sewing, buttoning, clamping, zipping, and/or bonding in any other manner.

Although the present invention has been described with reference to preferred embodiments, workers who are skilled in the art will recognize that changes may be made in form and detail without departing from the spirit and scope of the present invention.

What is claimed is:

1. A head support device comprising:
a seat-back securing piece including a securing adhesive front surface and a securing adhesive backing, the securing seat-back piece positionable and securable to a top portion of a seat; and
a headband wearable around a user's head, structured to comfortably fit above the user's ears, across the user's forehead, and around a back of the user's head, wherein the headband includes side adhesive surfaces, and includes a headband adhesive rear surface attachable to the securing adhesive front surface of the securing seat-back piece, thereby forming a secure contact between the headband and securing seat-back piece.

2. A head support device comprising:
a seat-back securing piece including a securing adhesive front surface and a securing adhesive backing, the securing seat-back piece positionable and securable to a top portion of a seat;
a headband wearable around a user's head, structured to comfortably fit above the user's ears, across the user's forehead, and around a back of the user's head, wherein the headband includes side adhesive surfaces, and includes a headband adhesive rear surface attachable to the securing adhesive front surface of the securing seat-back piece, thereby forming a secure contact between the headband and securing seat-back piece; and
an elongate jaw band having adhesive ends, the jaw band structured to be placed under the user's chin with each of the adhesive ends attached to each of the side adhesive surfaces.

3. The device of claim 2, wherein:
the securing adhesive front surface, the headband adhesive rear surface, and the side adhesive surfaces are hook-and-loop materials; and
the securing adhesive backing of the securing seat-back piece is a removably adhesive material.

4. The device of claim 2, wherein:
the securing adhesive front surface is a hook-and-loop material;
the securing adhesive backing is a removably adhesive material; and
the securing seat-back piece is disposable.

5. The device of claim 2, wherein:
the headband is an elastic material and includes a covering comprised of one of cotton or terrycloth; and
the jaw band is an elastic material and includes a covering comprised of one of cotton or terrycloth.

6. The device of claim 2, wherein the jaw band is removably affixed to the headband.

7. The device of claim 2, wherein the securing seat-back piece is removably securable to the seat.

8. A head support device comprising:
a seat-back section, formed by material having a section adhesive surface and a section adhesive backing, the section adhesive backing configured to provide a non-resilient secure integral attachment to a seat;
a circular section of material forming a resilient attachment around a user's head; the circular section having a back adhesive surface and side adhesive surfaces, the circular section being secured around the user's head by it's resilient elasticity, wherein the circular section is secured to the non-resilient seat-back via its resilient elasticity and adhesive surfaces;
an elongate section of material forming a resilient attachment around the user's chin; the elongate section having two chin adhesive surfaces, one at each opposite end of the elongate section, wherein the elongate section is secured around the user's chin via its chin adhesive surfaces.

9. The device of claim 8, wherein:
the section adhesive backing is a removably adhesive material.

10. The device in claim 8, wherein:
the section adhesive surface, back adhesive surface, side adhesive surfaces, and chin adhesive surfaces are hook-and-loop materials.

11. The device in claim 8, wherein:
the section adhesive surface, back adhesive surface, side adhesive surfaces, and chin adhesive surfaces include a surface area sufficient to allow for a degree of placement variation of attachment points for fitting preference.

12. The device in claim 8, wherein:
the seat-back section is a hook-and-loop material including an adhesive backing;
the circular section is a cotton-covered, elastic material; and
the elongate section is a cotton-covered, elastic material.

13. The device in claim 8 wherein the circular section is removably affixed to the seat-back section.

14. The device in claim 8 wherein each opposite end of the elongate section is removably affixed to the side adhesive surfaces of the circular section

15. A head support device comprising:
a seat-back securing piece including a securing adhesive front surface and a securing adhesive backing, the securing seat-back piece positionable and securable to a top portion of a seat;
a headband wearable around a user's head, structured to comfortably fit above the user's ears, across the user's forehead, and around a back of the user's head, wherein the headband includes side adhesive surfaces, and includes a headband adhesive rear surface attachable to the securing adhesive front surface of the securing seat-back piece, thereby forming a secure contact between the headband and securing seat-back piece; and
a U-shaped piece of material, taken from the group of plastic, rubber, or other resilient material, into which the back of the user's head is placed, with the piece's rear, flat surface equipped with an adhering hook-and-loop type closure surface, and said surface being secured into place against the anchoring adhesive-backed hook-and-loop type closure piece.

16. The head support device of claim 15, further comprising:
an elongate jaw band having adhesive ends, the jaw band structured to be placed under the user's chin with each of the adhesive ends attached to each of the side adhesive surfaces.
